(19) United States
(12) Patent Application Publication
(10) Pub. No.: US 2023/0316209 A1
(43) Pub. Date: Oct. 5, 2023

(54) PREDICTING CAREGIVER RETENTION USING MACHINE LEARNING

(71) Applicant: MatrixCare, Inc., Bloomington, MN (US)

(72) Inventors: Kedar Mangesh KADAM, Nova Scotia (CA); Vivek KUMAR, Eden Prairie, MN (US); Keegan Duane DSOUZA, Ocala, FL (US); Adhiraj Ganpat PRAJAPATI, St. Paul, MN (US)

(21) Appl. No.: 18/179,096

(22) Filed: Mar. 6, 2023

Related U.S. Application Data
(60) Provisional application No. 63/396,057, filed on Aug. 8, 2022.
(60) Provisional application No. 63/325,979, filed on Mar. 31, 2022.

(51) Int. Cl. G06Q 10/0639; G16H 40/20

(52) U.S. Cl. CPC G06Q 10/06398; G16H 40/20

(57) ABSTRACT

Techniques for predicting caregiver retention using machine learning (ML) are discussed. These techniques include predicting an impact of one or more caregiver tasks on continued employment of the caregiver with a care provider, including determining a plurality of intermediate prediction scores relating to characteristics for the caregiver using one or more first ML models trained to determine intermediate prediction scores. The techniques further include determining a retention prediction for the caregiver using the plurality of intermediate prediction scores, including: generating the retention prediction by providing the plurality of intermediate prediction scores to a second ML model trained to determine the retention prediction based on intermediate prediction scores. The retention prediction is provided to an electronic system relating to the caregiver to improve treatment for a patient of the caregiver by: (i) increasing a likelihood of continued employment for the caregiver or (ii) identifying a replacement for the caregiver.

### CROSS-REFERENCE TO RELATED APPLICATIONS

This application claims priority to U.S. Provisional Patent Application No. 63/396,057, filed Aug. 8, 2022, and U.S. Provisional Patent Application No. 63/325,979, filed Mar. 31, 2022, the entire contents of which are incorporated herein by reference in their entirety.

### INTRODUCTION

Aspects of the present disclosure relate to artificial intelligence and healthcare, and more specifically, to predicting caregiver retention using machine learning (ML).

Caregivers are persons tasked with providing in-home and clinical healthcare to patients. The caregiver profession can be extremely challenging, and demanding, which can result in significant caregiver turnover. Caregiver turnover can result in worse outcomes for patients because of a lack of continuity in providing care as well as increased costs for care providers (e.g., because of loss of productivity, recruitment, training, and onboarding of caregivers). This can be especially true for caregivers with less experience or formal schooling, and in economic environments where employees have numerous career options. What is needed are improved techniques to accurately, and computationally efficiently, predict caregiver retention.

### SUMMARY

Embodiments include a method. The method includes predicting an impact of one or more caregiver tasks on continued employment of the caregiver with a care provider, including determining a plurality of intermediate prediction scores relating to characteristics for the caregiver using one or more first machine learning (ML) models trained to determine intermediate prediction scores. The method further includes determining a retention prediction for the caregiver using the plurality of intermediate prediction scores, including: generating the retention prediction by providing the plurality of intermediate prediction scores to a second ML model trained to determine the retention prediction based on intermediate prediction scores. The retention prediction is provided to an electronic system relating to the caregiver to improve treatment for a patient of the caregiver by at least one of: (i) increasing a likelihood of continued employment for the caregiver or (ii) identifying a replacement for the caregiver.

Embodiments further include an apparatus. The apparatus includes a memory, and a hardware processor communicatively coupled to the memory, the hardware processor configured to perform operations. The operations include predicting an impact of one or more caregiver tasks on continued employment of the caregiver with a care provider, including determining a plurality of intermediate prediction scores relating to characteristics for the caregiver using one or more first ML models trained to determine intermediate prediction scores. The operations further include determining a retention prediction for the caregiver using the plurality of intermediate prediction scores, including: generating the retention prediction by providing the plurality of intermediate prediction scores to a second ML model trained to determine the retention prediction based on intermediate prediction scores. The retention prediction is provided to an electronic system relating to the caregiver to improve treatment for a patient of the caregiver by at least one of: (i) increasing a likelihood of continued employment for the caregiver or (ii) identifying a replacement for the caregiver.

Embodiments further include a non-transitory computer-readable medium including instructions that, when executed by a processor, cause the processor to perform operations. The operations include predicting an impact of one or more caregiver tasks on continued employment of the caregiver with a care provider, including determining a plurality of intermediate prediction scores relating to characteristics for the caregiver using one or more first ML models trained to determine intermediate prediction scores. The operations further include determining a retention prediction for the caregiver using the plurality of intermediate prediction scores, including: generating the retention prediction by providing the plurality of intermediate prediction scores to a second ML model trained to determine the retention prediction based on intermediate prediction scores. The retention prediction is provided to an electronic system relating to the caregiver to improve treatment for a patient of the caregiver by at least one of: (i) increasing a likelihood of continued employment for the caregiver or (ii) identifying a replacement for the caregiver.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts a computing environment for predicting caregiver retention using ML, according to one embodiment.

FIG. 2 depicts a block diagram for a prediction controller for predicting caregiver retention using ML, according to one embodiment.

FIG. 3A is a flowchart illustrating predicting caregiver retention using ML, according to one embodiment.

FIG. 3B is a flowchart illustrating generating intermediate prediction scores for predicting caregiver retention using ML, according to one embodiment.

FIG. 4A illustrates determining an intermediate prediction score using ML, according to one embodiment.

FIG. 4B is a flowchart illustrating training an ML Model to determine an intermediate prediction score, according to one embodiment.

FIGS. 5A-C depict example intermediate prediction data for determining an intermediate prediction score using ML, according to one embodiment.

FIG. 6 depicts example progress note data for determining an intermediate prediction score using ML, according to one embodiment.

FIG. 7 depicts example task data for determining an intermediate prediction score using ML, according to one embodiment.

FIG. 8A illustrates predicting caregiver retention using an ML model, according to one embodiment.

FIG. 8B is a flowchart illustrating training an ML Model to predict caregiver retention, according to one embodiment.

FIG. 9 depicts example historical caregiver outcome data for predicting caregiver retention using an ML model, according to one embodiment.

FIG. 10 depicts pre-processing textual data using natural language processing (NLP), according to one embodiment.

FIG. 11 depicts predicted caregiver retention outcomes using ML, according to one embodiment.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved prediction of caregiver retention using ML. As discussed above, caregiver retention is a challenging, and important, problem. High caregiver turnover, or unexpected caregiver turnover, can be detrimental to patient outcomes and expensive and challenging for care facilities and entities employing caregivers. Further, electronic scheduling systems can be used to schedule caregivers, but it can be extremely difficult and computationally expensive to electronically identify an appropriate caregiver schedule (e.g., for a healthcare facility).

In aspects described herein, caregiver retention can be predicted automatically, using a trained ML model. For example, one or more ML models can be trained to identify intermediate prediction scores. This can include a facility score (e.g., using facility characteristics), a patient score (e.g., using patient characteristics), a caregiver performance score (e.g., using caregiver performance characteristics), a progress note score (e.g., using progress note characteristics), or a task score (e.g., using task characteristics). Each of these can be generated using a trained ML model (e.g., trained using historical retention data), and can be used to identify a likelihood that a particular characteristic or category of characteristics is contributing to lowered retention for caregivers.

In an embodiment, these scores can be used, along with other data (e.g., caregiver data, compatibility data, and any other suitable data) by a trained ML model to predict caregiver retention. For example, the ML model can predict the likelihood that particular caregivers, or particular classes of caregivers, will leave their positions over a given time horizon. As another example, the ML model can predict which factors (e.g., which tasks, facility characteristics, caregiver characteristics, or other factors) contribute most significantly to the likelihood that the caregivers will leave their positions. As a final example, the ML model can predict recommended changes to caregiver tasks, care facilities, and other aspects of patient care to reduce the chances that caregivers will leave their positions. Further, the intermediate prediction scores can be analyzed using heuristics or statistical techniques, without an ML model, to predict caregiver retention.

Aspects described herein provide significant advantages over conventional systems. For example, predicting factors that lower caregiver retention, and predicting recommended changes to reduce caregiver turnover, significantly improves patient treatment outcomes. Low caregiver retention can lead to inexperienced or inappropriate caregivers working with patients, and can lead to worse patient outcomes and lowered patient satisfaction. Further, predicting caregiver retention allows a care provider (e.g., an employer of caregivers) to more accurately predict needed resources and hire appropriately. This can ensure that patients have sufficient care, at all times, and can reduce the overhead expenses and costs for the care provider.

Using a trained ML model to perform these predictions also provides a significant technical advantage. For example, in an embodiment some aspects of retention could potentially be analyzed using a specific rubric or algorithm with pre-defined rules. But this may be computationally expensive, because a very large number of rules would be needed and parsing and following the rules is computationally expensive. Further, using pre-defined rules would require this computationally expensive analysis be done at the time of the prediction, when a rapid response is likely to be needed (e.g., so that the caregiver retention can be identified quickly, and new caregivers hired, or so that factors detrimental to caregiver retention can be improved quickly). Predicting retention automatically using a trained ML model, by contrast, is significantly less computationally expensive at the time the prediction generated. For example, the ML model can be trained up-front during a training phase, when rapid response is not necessary and computational resources are readily available. The trained ML model can then be used to rapidly, and computationally cheaply, predict retention.

As another example, predicting retention using a trained ML model, based on one or more intermediate prediction scores, or other data, provides for a more accurate and well-defined result. In an embodiment, a care provider could manually predict the expected retention. But this leaves the risk of human error, and a lack of certainty in the accuracy of the prediction. Predicting retention using a trained ML model can both lessen the risk of human error, and provide more certainty in the level of accuracy of the prediction. Further, the prediction can itself be reviewed and refined by a care provider. This provides a starting point for the care provider with a more certain level of accuracy, and reduces the burden on the care provider to generate the prediction themselves. This is especially true because the care provider will almost certainly not have access or knowledge of all of the historical data, described further below, that can be considered at instant using one or more of the ML techniques described below.

As another example, one or more techniques described below provide an improvement to electronic scheduling systems. As noted above, electronically generating a schedule for caregivers is difficult. For example, electronically scheduling a large number of caregivers, without predicting retention as described herein, can be computationally expensive because it can require generating a very large number of schedules and undergoing trial and error to identify the preferred or optimal schedule to maintain retention of caregivers. It can also be inaccurate, and schedules must be re-generated each time a caregiver leaves. One or more techniques described herein improve this, by predicting retention. As described below, in an embodiment the retention prediction can be automatically provided to an electronic scheduling system (e.g., of a caregiver or healthcare facility), and the electronic scheduling system can use the prediction to improve both the efficiency (e.g., reduce the computational burden) and quality of the electronic scheduling.

### Example Computing Environment

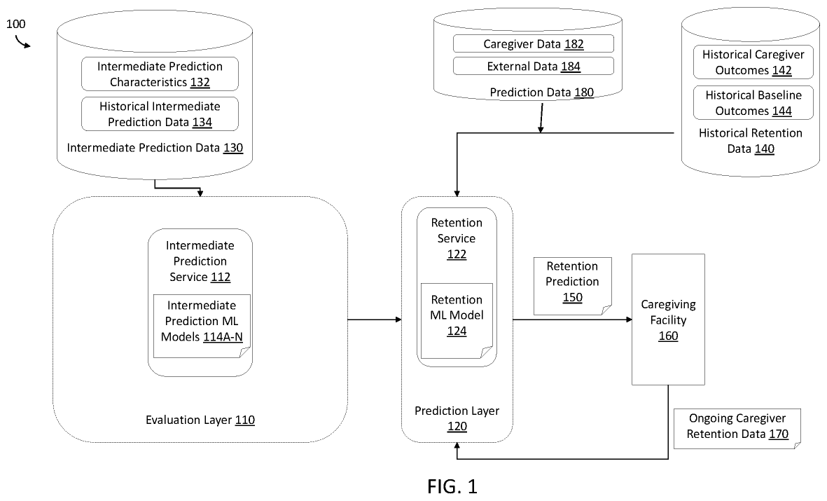

FIG. 1 depicts a computing environment 100 for predicting caregiver retention using ML, according to one embodiment. In an embodiment, intermediate prediction data are provided to an evaluation layer 110. The evaluation layer 110 can use one or more ML models (e.g., one or more intermediate prediction ML models 114A-N) to determine effects of a particular intermediate prediction on retention of caregivers. This can include a facility score (e.g., using facility characteristics), a patient score (e.g., using patient characteristics), a caregiver performance score (e.g., using caregiver performance characteristics), a progress note score (e.g., using progress note characteristics), or a task score (e.g., using task characteristics).

In an embodiment, the intermediate prediction data 130 are provided to the evaluation layer 110 using a suitable communication network. In an embodiment, the intermediate prediction data includes intermediate prediction characteristics (e.g., data used for inference by an ML model) and historical intermediate prediction data (e.g., historical data used for training an ML model). The intermediate prediction data 130 can be stored in one or more suitable electronic databases (e.g., a relational database, a graph database, or any other suitable database) or other electronic repositories (e.g., a cloud storage location, an on-premises network storage location, or any other suitable electronic repository), and can be provided to the evaluation layer 110 using any suitable communication network, including the Internet, a wide area network, a local area network, or a cellular network, and can use any suitable wired or wireless communication technique (e.g., WiFi or cellular communication).

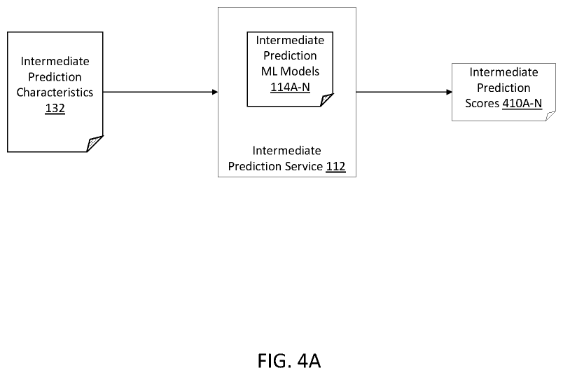

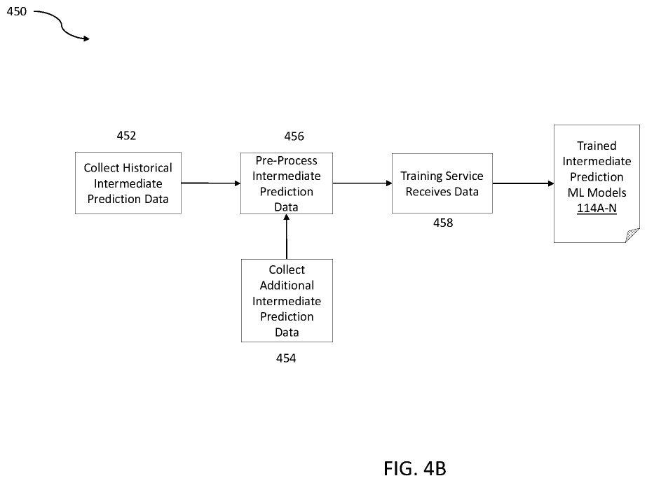

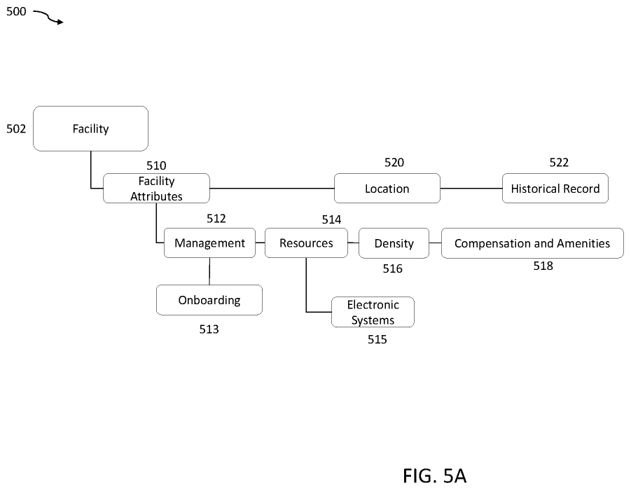

For example, an intermediate prediction service 112 can use one or more intermediate prediction ML models 114A-N to determine intermediate prediction scores using the intermediate prediction data 130. In an embodiment, the intermediate prediction scores can indicate the predicted effect of a wide variety of caregiver related data on caregiver retention. This is discussed further, below, with regard to FIGS. 4A-7.

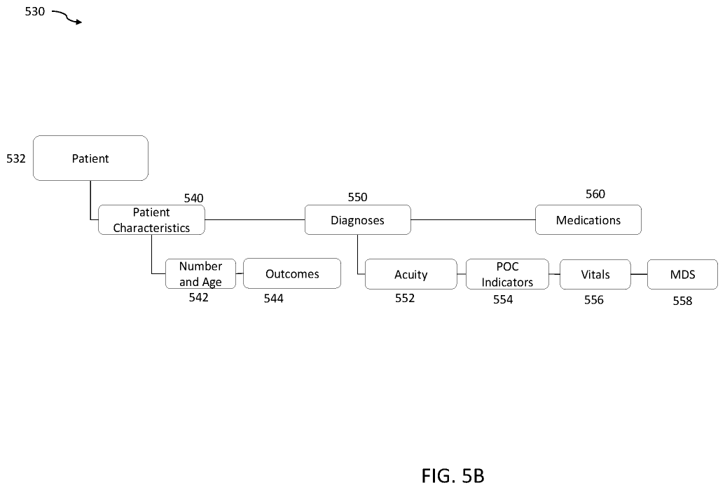

For example, the intermediate prediction scores can include a facility score, which can indicate the predicted effect of the facility on caregiver retention (e.g., whether the facility increases retention, decreases retention, or has a neutral effect). The facility characteristics can include characteristics of patients at the facility (e.g., the number of patients and acuity of patient health conditions at the facility), attributes of the facility itself (e.g., management attributes, available resources, density of patients, and compensation and amenities for caregivers), and location. This is discussed further below with regard to FIG. 5. The facility data can indicate historical retention rates for caregivers at the facility (e.g., over time, based on class of caregiver, or using any other suitable criteria).

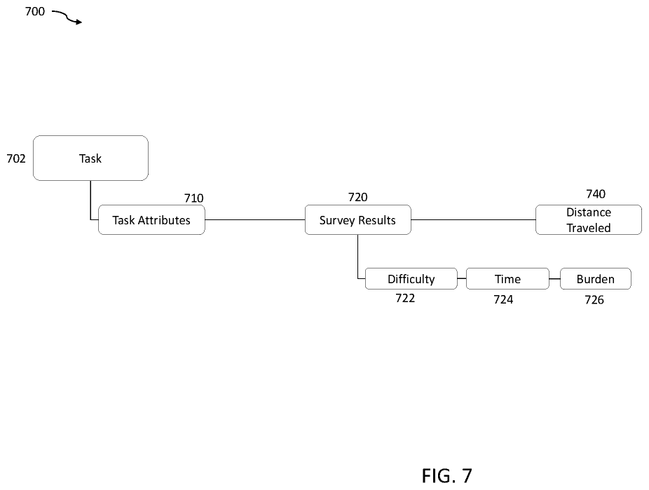

As another example, the intermediate prediction scores can include a task score, which can indicate the predicted effect of a given caregiver task (e.g., a task performed by a caregiver as part of their duties), or a given collection of tasks, on caregiver retention (e.g., whether the task increases retention, decreases retention, or has a neutral effect). The task characteristics can include attributes of the task (e.g., the type or classification of task, the caregiver skill required for the task, or any other suitable attributes of the task), survey results for the task (e.g., results of one or more surveys provided to caregivers assessing the task), and distance traveled by the caregiver to perform the task (e.g., distance traveled within a facility). This is discussed further below with regard to FIG. 7. The task data can indicate historical retention rates for caregivers that regularly perform the task or collection of tasks (e.g., over time, based on class of caregiver, or using any other suitable criteria).

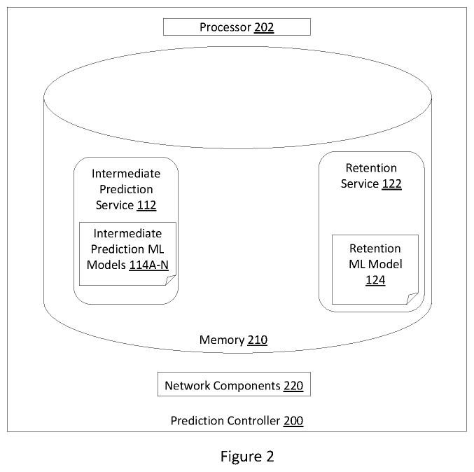

In an embodiment, as discussed below with regard to FIG. 2, the intermediate prediction service 112 can be a computer software service implemented in a suitable controller (e.g., the prediction controller 200 illustrated in FIG. 2) or combination of controllers. In an embodiment, the evaluation layer 110 provides the intermediate prediction scores, and any other suitable data (e.g., a combined score) to a prediction layer 120. The prediction layer 120 includes a retention service 122 and a retention ML model 124. As discussed below with regard to FIG. 2, the intermediate prediction service 112 and retention service 122 can each be a computer software service implemented in a suitable controller (e.g., the prediction controller 200 illustrated in FIG. 2) or combination of controllers. In an embodiment the evaluation layer 110 and the prediction layer 120, and the intermediate prediction service 112 and retention service 122, can be implemented using any suitable combination of physical compute systems, cloud compute nodes and storage locations, or any other suitable implementation. For example, the evaluation layer 110 and prediction layer 120 could each be implemented using a server or cluster of servers. As another example, the evaluation layer 110 and prediction layer 120 can be implemented using a combination of compute nodes and storage locations in a suitable cloud environment. For example, one or more of the components of the evaluation layer 110 and prediction layer 120 can be implemented using a public cloud, a private cloud, a hybrid cloud, or any other suitable implementation.

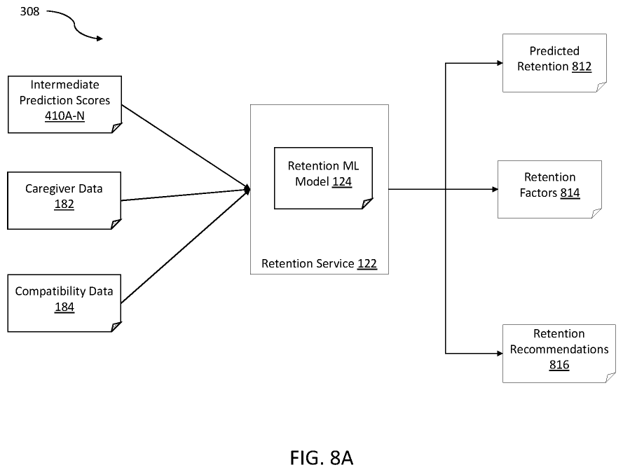

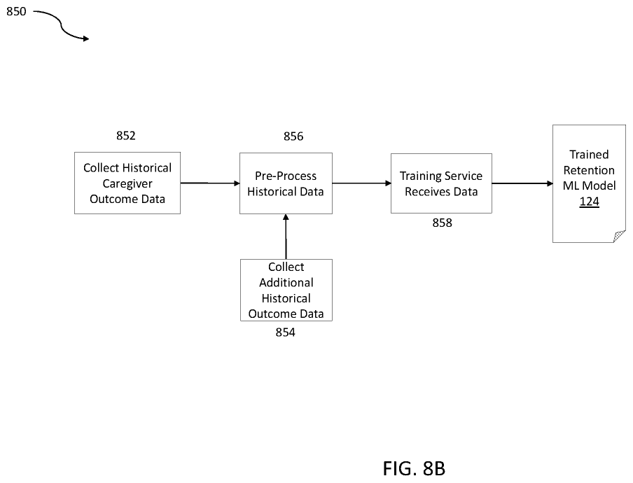

In an embodiment, the retention service 122 uses the retention ML model 124 to predict retention data using the data from the evaluation layer 110 (e.g., intermediate prediction scores), and additional prediction data 180 (e.g., caregiver data 182 and compatibility data 184). For example, the retention ML model 124 can predict expected retention for a given caregiver or group of caregivers, can predict retention factors likely to impact retention for caregivers, and can predict recommended changes to reduce the likelihood of caregiver turnover. This is discussed further below with regard to FIGS. 8A-B. These are merely example, and the retention service 122 can use the retention ML model 124 to predict any suitable retention information.

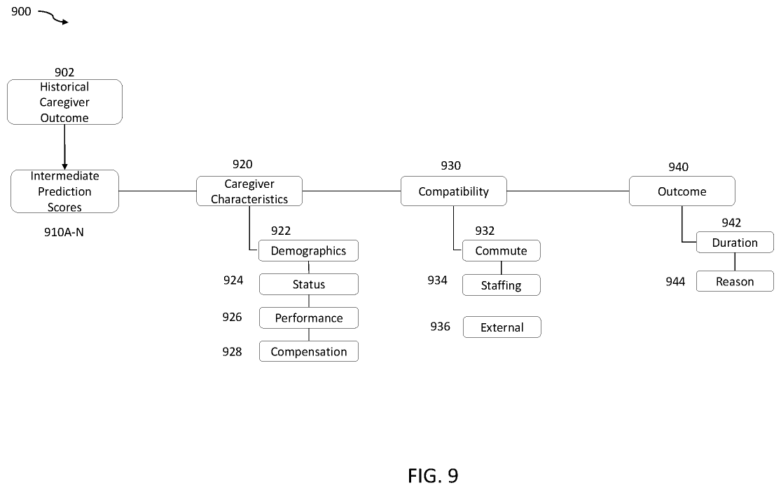

Further, the retention service 122 can use historical retention data 140 to train the retention ML model 124. The historical retention data 140 can include historical caregiver outcomes 142 and historical baseline outcomes 144. This is discussed further below, with regard to FIGS. 8B and 9. Further, in an embodiment, the historical retention data 140 has had any personally identifying information (e.g., caregiver or patient information) removed.

In an embodiment, the prediction data 180 and the historical retention data 140 are provided to the prediction layer 120 using a suitable communication network. For example, the prediction data 180 and the historical retention data 140 can be stored in one or more suitable electronic databases (e.g., a relational database, a graph database, or any other suitable database) or other electronic repositories (e.g., a cloud storage location, an on-premises network storage location, or any other suitable electronic repository). The prediction data 180 and the historical retention data 140 can be provided from the respective electronic repositories to the prediction layer 120 using any suitable communication network, including the Internet, a wide area network, a local area network, or a cellular network, and can use any suitable wired or wireless communication technique (e.g., WiFi or cellular communication).

For example, the retention service 122 can provide a retention prediction 150 (e.g., any, or all, of predicted retention for a given caregiver or caregivers, retention factors, or retention recommendations) to a caregiving facility 160 (e.g., an individual caregiving facility, an entity managing caregiving facilities, another healthcare entity, or any other suitable entity). In an embodiment, the retention prediction 150 can include textual or graphical information to identify expected retention for a given caregiver or group of caregivers (e.g., providing a list of caregivers with risk of turnover, identifying caregivers most at risk of turnover, identifying an expected rate of retention for particular caregivers or groups of caregivers, or providing any other suitable information), can predict retention factors likely to impact retention for caregivers (e.g., identifying factors predicted to impact retention for particular caregivers or groups of caregivers, identifying categories of factors predicted to impact retention for particular caregivers or groups of caregivers, or providing any other suitable information) and can predict recommended changes to reduce the likelihood of caregiver turnover (e.g., providing recommendations to improve retention for particular caregivers or groups of caregivers, or providing any other suitable information).

The caregiving facility 160 can use the retention prediction 150 to plan for possible turnover, identify likely factors in caregiver turnover, implement retention recommendations, or to take any other suitable action. Further, the caregiving facility 160 can provide ongoing caregiver retention data 170 to the prediction layer 120. The retention service 122 can use the ongoing caregiver retention data 170 to update (e.g., re-train) the retention ML model. This can improve the accuracy of the retention prediction 150 from the retention ML model 124.

FIG. 2 depicts a block diagram for a prediction controller for predicting caregiver retention using ML, according to one embodiment. The controller 200 includes a processor 202, a memory 210, and network components 220. The memory 210 may take the form of any non-transitory computer-readable medium. The processor 202 generally retrieves and executes programming instructions stored in the memory 210. The processor 202 is representative of a single central processing unit (CPU), multiple CPUs, a single CPU having multiple processing cores, graphics processing units (GPUs) having multiple execution paths, and the like.

The network components 220 include the components necessary for the controller 200 to interface with a suitable communication network (e.g., a communication network interconnecting various components of the computing environment 100 illustrated in FIG. 1, or interconnecting the computing environment 100 with other computing systems). For example, the network components 220 can include wired, WiFi, or cellular network interface components and associated software. Although the memory 210 is shown as a single entity, the memory 210 may include one or more memory devices having blocks of memory associated with physical addresses, such as random access memory (RAM), read only memory (ROM), flash memory, or other types of volatile and/or non-volatile memory.

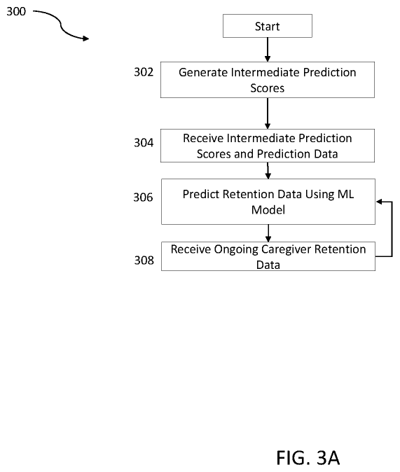

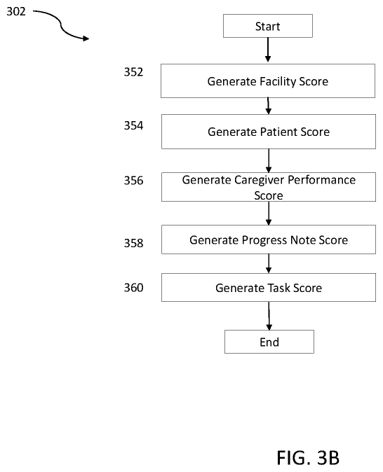

The memory 210 generally includes program code for performing various functions related to use of the prediction controller 200. The program code is generally described as various functional “applications” or “modules” within the memory 210, although alternate implementations may have different functions and/or combinations of functions. Within the memory 210, the intermediate prediction service 112 facilitates predicting intermediate retention scores using one or more intermediate prediction ML models 114A-N. This is discussed further below with regard to FIGS. 4A-B and 5. The retention service 122 facilitates predicting retention information (e.g., any, or all, of predicted retention for a given caregiver or caregivers, retention factors, or retention recommendations), using the retention ML model 124. This is discussed further below with regard to FIGS. 3, 8A-B, and 9.

While the controller 200 is illustrated as a single entity, in an embodiment, the various components can be implemented using any suitable combination of physical compute systems, cloud compute nodes and storage locations, or any other suitable implementation. For example, the controller 200 could be implemented using a server or cluster of servers. As another example, the controller 200 can be implemented using a combination of compute nodes and storage locations in a suitable cloud environment. For example, one or more of the components of the controller 200 can be implemented using a public cloud, a private cloud, a hybrid cloud, or any other suitable implementation.

Although FIG. 2 depicts the intermediate prediction service 112, the retention service 122, the one or more intermediate prediction ML models 114A-N, and the retention ML model 124, as being mutually co-located in memory 210, that representation is also merely provided as an illustration for clarity. More generally, the controller 200 may include one or more computing platforms, such as computer servers for example, which may be co-located, or may form an interactively linked but distributed system, such as a cloud-based system, for instance. As a result, processor 202 and memory 210 may correspond to distributed processor and memory resources within the computing environment 100. Thus, it is to be understood that any, or all, of the intermediate prediction service 112, the retention service 122, the intermediate prediction ML models 114A-N, and the retention ML model 124 may be stored remotely from one another within the distributed memory resources of the computing environment 100.

FIG. 3 is a flowchart 300 illustrating predicting caregiver retention using ML, according to one embodiment.

At block 302 an intermediate prediction service (e.g., the intermediate prediction service 112 illustrated in FIGS. 1-2) generates intermediate prediction scores. For example, the intermediate prediction service can use one or more intermediate prediction ML models (e.g., the intermediate prediction ML models 114A-N illustrated in FIGS. 1-2) to predict intermediate prediction scores.

In an embodiment, the one or more intermediate prediction ML models are trained ML models (e.g., a trained deep neural network (DNN), regression model, or any other suitable supervised ML model). For example, the one or more intermediate prediction ML models can be a multilayer perception (MLP) neural network with the use of one or more hidden layers, trained using suitable training data. This is discussed further below with regard to FIG. 4B.

In an embodiment, the intermediate prediction service can use the one or more intermediate prediction ML models to predict intermediate prediction scores (e.g., during inference). This is discussed further below with regard to FIG. 3B. F or example, the intermediate prediction service can predict any, or all, of a facility score (e.g., using facility characteristics), a patient score (e.g., using patient characteristics), a caregiver performance score (e.g., using caregiver performance characteristics), a progress note score (e.g., using progress note characteristics), or a task score (e.g., using task characteristics).

In an embodiment, the intermediate prediction scores can indicate a likelihood that a particular characteristic or collection of characteristics contributes to retention, or turnover, of caregivers working at that facility. As another example, the intermediate prediction scores can indicate a magnitude of the impact of the characteristics on the retention, or turnover, of caregivers working at the facility. In an embodiment, the intermediate prediction scores score is a number, or tuple of numbers, indicating one or more of likelihood and magnitude that the intermediate characteristics contribute to retention. This is merely an example, and the intermediate prediction scores can be any suitable value or collection of values (e.g., one or more Boolean values, textual values, or any other suitable values).

Further, in an embodiment, the one or more intermediate prediction ML models can predict the intermediate retention scores score using one or more intermediate prediction characteristics (e.g., the intermediate prediction characteristics 132 illustrated in FIG. 1). The intermediate prediction characteristics can include any suitable characteristics. This is discussed further below with regard to FIGS. 5-7.

At block 304 a retention service (e.g., the retention service 122 illustrated in FIGS. 1-2) receives intermediate retention scores, and prediction data. As discussed further below with regard to block 308, in an embodiment, the retention service uses a retention ML model (e.g., the retention ML model 124 illustrated in FIGS. 1-2) to predict retention data for a given caregiver or collection of caregivers (e.g., any, or all, of predicted retention, retention factors, or retention recommendations). The retention ML model can predict retention data using intermediate retention scores.

The retention ML model can further use additional prediction data (e.g., prediction data 180 illustrated in FIG. 1). This additional prediction data can include either, or both, of caregiver data (e.g., the caregiver data 182 illustrated in FIG. 1), compatibility data (e.g., the compatibility data 184 illustrated in FIG. 1), and any other suitable data. For example, the additional prediction data can include characteristics of the caregiver (e.g., experience, skills and training, past performance, commute distance, and any other suitable characteristics) and compatibility characteristics (e.g., employment and economic information for the caregiver's local region).

At block 306, the retention service predicts retention data using the retention ML model. In an embodiment, the retention ML model is a trained ML model (e.g., a trained DNN or any other suitable supervised ML model). For example, the retention ML model can be an MLP neural network with the use of one or more hidden layers, trained using historical retention data (e.g., the historical retention data 140 illustrated in FIG. 1) or any other suitable data. This is discussed further below with regard to FIG. 8B. The historical retention data can include historical caregiver outcomes (e.g., the historical caregiver outcomes 142 illustrated in FIG. 1). This is discussed further below with regard to FIG. 9. The historical retention data can further include historical baseline outcomes (e.g., the historical baseline outcomes 144 illustrated in FIG. 1). This can include baseline retention outcomes for caregivers (e.g., for a region, category of caregiver, type of facility, or based on any other criteria). Further, in an embodiment, the historical retention data is data collected at different snapshots of time. For example, the historical retention data can be collected using a lookback period (e.g., one month prior to the snapshot).

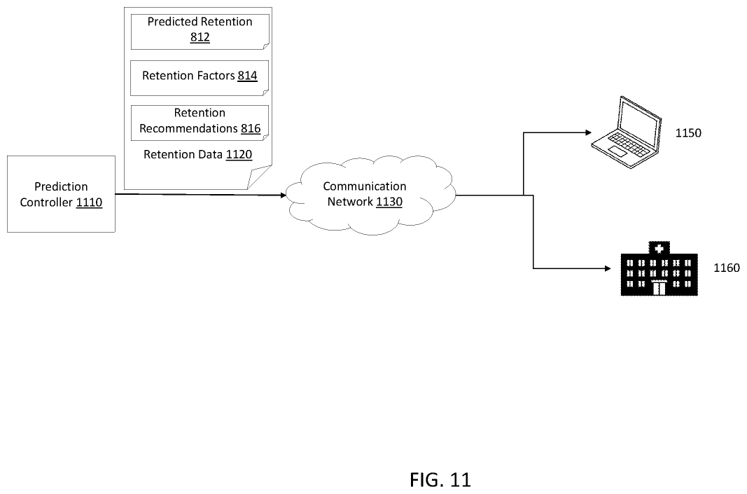

In an embodiment, the retention service can use the retention ML model to predict retention data (e.g., during inference). This is discussed further below with regard to FIG. 8A. For example, the retention ML model can predict retention data for a given caregiver or collection of caregivers (e.g., any, or all, of predicted retention, retention factors, or retention recommendations). This is discussed further below with regard to FIG. 11. This is merely an example, and the retention ML model can predict any suitable retention data.

At block 308 the retention service receives ongoing caregiver retention data. For example, the retention service can receive additional data reflecting retention, or turnover, of caregivers (e.g., at a particular facility performing particular tasks). In an embodiment, the retention service can use the ongoing data to further refine the prediction of retention data. For example, the retention service can provide the ongoing data to the facility evaluation service, which can use the data to update (e.g., re-train) the facility evaluation ML model. As another example, the retention service can provide the ongoing data to the task evaluation service, which can use the data to update (e.g., re-train) the task evaluation ML model. As another example, the retention service can use the ongoing data to update (e.g., re-train) the retention ML model).

FIG. 3B is a flowchart illustrating generating intermediate prediction scores for predicting caregiver retention using ML, according to one embodiment. In an embodiment, FIG. 3B corresponds with block 302 illustrated in FIG. 3A, above. At block 352, an intermediate prediction service (e.g., the intermediate prediction service 112 illustrated in FIGS. 1-2) generates a facility score. In an embodiment, the intermediate prediction service predicts a facility score from facility characteristics using an ML model. This is one example of using an intermediate prediction ML model (e.g., a facility ML model) to generate an intermediate prediction score (e.g., a facility score) from intermediate prediction characteristics (e.g., facility characteristics), as illustrated below in relation to FIG. 4A. As illustrated below in relation to FIG. 4B, this intermediate prediction ML model (e.g., a facility ML model) can be a suitable trained ML model. The facility score can then be used to predict caregiver attrition, as discussed above in relation to block 304 in FIG. 3.

For example, the facility characteristics can include characteristics of the facility (e.g., management attributes, onboarding information, available resources, electronic system information, density of patients, and compensation and amenities for caregivers), location, a historical record for the facility (e.g., historical falls or patient outcomes), and any other suitable data. This is discussed further below with regard to FIG. 5A.

In an embodiment, facility characteristics can be used to assist in predicting caregiver retention. An intermediate ML model can be trained to predict how, and whether, facility characteristics impact predictions of caregiver attrition. As one example, differences between facility characteristics for a given facility and baseline facility characteristics (e.g., characteristics of highly rated facilities or similarly rated facilities), changes in facility characteristics over time, or any other suitable aspect of facility characteristics, can indicate a change in the likelihood of attrition for the caregiver. This is merely an example.

At block 354 the intermediate prediction service generates a patient score. In an embodiment, the intermediate prediction service predicts a patient score from patient characteristics using an ML model. This is one example of using an intermediate prediction ML model (e.g., a patient retention ML model) to generate an intermediate prediction score (e.g., a patient score) from intermediate prediction characteristics (e.g., patient characteristics), as illustrated below in relation to FIG. 4A. As illustrated below in relation to FIG. 4B, this intermediate prediction ML model (e.g., a patient retention ML model) can be a suitable trained ML model. The patient retention score can then be used to predict caregiver attrition, as discussed above in relation to block 304 in FIG. 3.

For example, the patient characteristics can include characteristics of patients at the facility (e.g., the number of patients and outcomes for patients at the facility), diagnoses for the patients (e.g., acuity of patients at the facility, point of care (POC) indicators, vitals information), medications for the patients, and any other suitable information. This is discussed further below with regard to FIG. 5B.

In an embodiment, patient characteristics can be used to assist in predicting caregiver retention. An intermediate ML model can be trained to predict how, and whether, patient characteristics impact predictions of caregiver attrition. As one example, differences between patient characteristics for a given facility and baseline patient characteristics (e.g., characteristics of patients at other facilities or average patient characteristics), changes in patient characteristics over time, or any other suitable aspect of patient characteristics, can indicate a change in the likelihood of attrition for the caregiver. This is merely an example.

At block 356 the intermediate prediction service generates a caregiver performance score. In an embodiment, the intermediate prediction service predicts a caregiver performance score from caregiver performance characteristics using an ML model. This is one example of using an intermediate prediction ML model (e.g., a caregiver performance characteristic ML model) to generate an intermediate prediction score (e.g., a caregiver performance score) from intermediate prediction characteristics (e.g., caregiver performance characteristics), as illustrated below in relation to FIG. 4A. As illustrated below in relation to FIG. 4B, this intermediate prediction ML model (e.g., a caregiver performance ML model) can be a suitable trained ML model. The caregiver performance score can then be used to predict caregiver attrition, as discussed above in relation to block 304 in FIG. 3.

For example, the caregiver performance characteristics can include caregiver errors (e.g., missed documentation, judgment errors, and formal reprimands), caregiver work characteristics (e.g., shift schedule, number of facilities worked at, accolades), and any other suitable information. This is discussed further below with regard to FIG. 5C.

In an embodiment, caregiver performance characteristics can be used to assist in predicting caregiver retention. An intermediate ML model can be trained to predict how, and whether, caregiver performance characteristics impact predictions of caregiver attrition. As one example, differences between caregiver performance characteristics for a given caregiver and baseline caregiver characteristics (e.g., performance characteristics of other caregivers at a given facility or group of facility or average caregiver performance characteristics), changes in caregiver performance characteristics over time, or any other suitable aspect of caregiver performance characteristics, can indicate a change in the likelihood of attrition for the caregiver. This is merely an example.

At block 358, the intermediate prediction generates a progress notes score. In an embodiment, the intermediate prediction service predicts a progress notes score from progress notes characteristics using an ML model. This is one example of using an intermediate prediction ML model (e.g., a progress notes prediction ML model) to generate an intermediate prediction score (e.g., a progress notes prediction score) from intermediate prediction characteristics (e.g., progress notes characteristics), as illustrated below in relation to FIG. 4A. As illustrated below in relation to FIG. 4B, this intermediate prediction ML model (e.g., a progress notes prediction ML model) can be a suitable trained ML model.

For example, a caregiver can maintain progress notes describing patient interactions and treatment progress. These notes can be generated from textual descriptions written by the caregiver, generated from selections of pre-written descriptions accessible to the caregiver using a suitable user interface (e.g., check boxes, drop downs, or any other suitable user interface elements), or generated from any other suitable source.

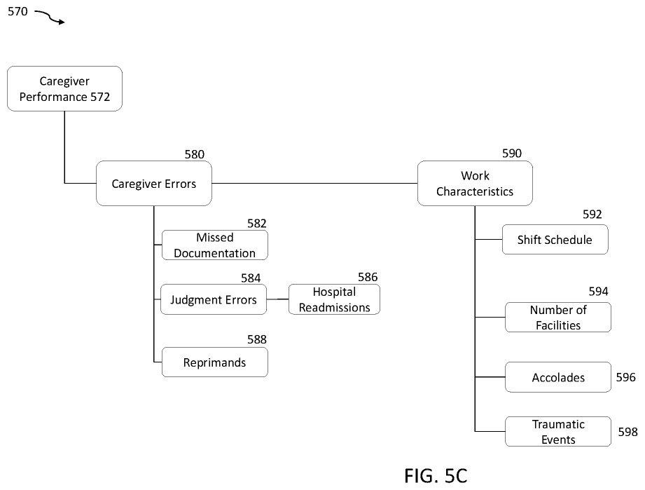

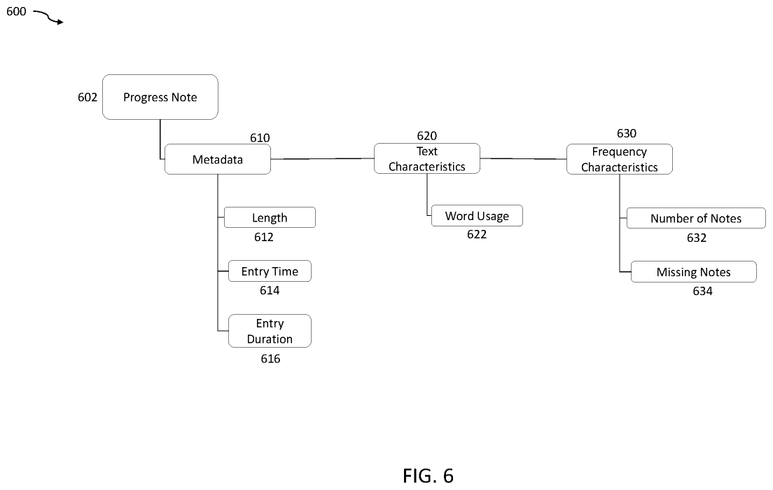

In an embodiment, characteristics of progress notes can be used to assist in predicting caregiver retention. These characteristics can include, for example, progress note length, time of entry of progress notes, usage of words in progress notes, a number of progress notes (e.g., for a given shift or time period), missing progress notes, and any other suitable characteristics. This is illustrated further, below, with regard to FIG. 6.

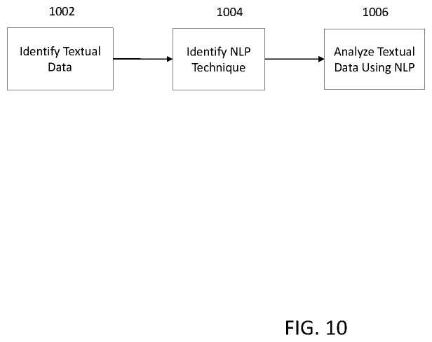

In this example, an intermediate ML model can be trained to predict how, and whether, progress note characteristics impact predictions of caregiver attrition. As one example, changes in progress note characteristics for a caregiver over time, or differences between progress note characteristics for a given caregiver and baseline expected progress note characteristics, can indicate a change in the likelihood of attrition for the caregiver. A caregiver that decreases the length of notes, changes the time(s) at which they enter notes, starts missing progress notes, or starts using less sophisticated language in progress notes, might be overburdened or otherwise more likely to leave a position. By contrast, a caregiver that increases the length of notes, stops missing progress notes, or starts using more sophisticated language in progress notes, might be less likely to leave a position. In an embodiment, natural language process (NLP) techniques can be used to identify progress note characteristics from textual progress notes. This is discussed further, below, with regard to FIG. 10.

At block 360 the intermediate prediction service generates a task score. In an embodiment, the intermediate prediction service predicts a task score from task characteristics using an ML model. This is one example of using an intermediate prediction ML model (e.g., a task ML model) to generate an intermediate prediction score (e.g., a task score) from intermediate prediction characteristics (e.g., task characteristics), as illustrated below in relation to FIG. 4A. As illustrated below in relation to FIG. 4B, this intermediate prediction ML model (e.g., a task ML model) can be a suitable trained ML model. The task score can then be used to predict caregiver attrition, as discussed above in relation to block 304 in FIG. 3.

For example, the task characteristics can include attributes of the task (e.g., the type or classification of task, the caregiver skill required for the task, or any other suitable attributes of the task), survey results for the task (e.g., results of one or more surveys provided to caregivers assessing the task), and distance traveled by the caregiver to perform the task (e.g., distance traveled within a facility). This is discussed further below with regard to FIG. 7.

In an embodiment, task characteristics can be used to assist in predicting caregiver retention. An intermediate ML model can be trained to predict how, and whether, task characteristics impact predictions of caregiver attrition. As one example, differences between task characteristics for a given caregiver or facility and baseline task characteristics (e.g., characteristics tasks for other caregivers or facilities), changes in task characteristics over time, or any other suitable aspect of task characteristics, can indicate a change in the likelihood of attrition for the caregiver. This is merely an example.

### Example of Predicting Intermediate Retention Scores

FIG. 4A illustrates determining an intermediate prediction score using ML, according to one embodiment. In an embodiment, FIG. 4A provides one example of predicting any of the intermediate prediction scores discussed above in relation to FIG. 3B. Intermediate prediction characteristics 132 are provided to an intermediate prediction service 112 and one or more intermediate prediction ML models 114A-N. In an embodiment, the intermediate prediction characteristics 132 include any of the data described below in relation to FIGS. 5A-7, or any other suitable data.

In an embodiment, the intermediate prediction service 112 can use the one or more intermediate prediction ML models 114A-N to predict one or more intermediate prediction scores 410A-N (e.g., during inference). For example, each of the one or more intermediate prediction scores 410A-N can indicate a likelihood that characteristics contribute to retention, or turnover, of caregivers. As another example, each of the one or more intermediate prediction scores 410A-N can indicate a magnitude of the impact of the characteristics on the retention, or turnover, of caregivers.

In an embodiment, each of the one or more intermediate prediction scores 410A-N is a number, or tuple of numbers, indicating one or more of likelihood and magnitude that the corresponding characteristics contribute to retention. For example, the intermediate prediction score can be a tuple of two numbers indicating the likelihood that the corresponding characteristics contribute to turnover (or retention) and the magnitude of the contribution. This is merely an example, and the intermediate prediction score can be any suitable value or collection of values (e.g., one or more Boolean values, textual values, or any other suitable values).

In an embodiment, each of the intermediate prediction ML models 114A-N can be any suitable ML model. For example, a neural network can be used (e.g., a DNN or any other suitable neural network) or a non neural-network can be used (e.g., a logistic regression model, a linear regression model, a decision tree, a support vector machine, a Bayesian network, a gradient boosting machine, or any other suitable non-neural network ML model).

### Example of Training an Intermediate Prediction ML Model

FIG. 4B is a flowchart 450 illustrating training an ML model to determine an intermediate prediction score (e.g., any of the intermediate prediction scores illustrated in FIG. 3B, or any other suitable intermediate prediction score), according to one embodiment. At block 452, a training service (e.g., a human administrator or a software or hardware service) collects historical intermediate prediction data. For example, an intermediate prediction service (e.g., the intermediate prediction service 112 illustrated in FIGS. 1 and 2) can be configured to act as the training service and collect historical intermediate prediction data (e.g., the historical intermediate prediction data 134 illustrated in FIG. 1). The historical intermediate prediction data can include historical data relevant to the intermediate prediction at issue (e.g., facility data, patient data, caregiver data, progress note data, task data, or any other suitable data). In an embodiment, the historical intermediate prediction data is data collected at different snapshots of time. For example, the historical intermediate prediction data can be collected using a lookback period (e.g., one month prior to the snapshot). This is merely an example, and any suitable software or hardware service can be used (e.g., an intermediate prediction training service) and any suitable training data can be used.

At block 456, the training service (or other suitable service) pre-processes the collected historical intermediate prediction data. For example, the training service can create feature vectors reflecting the values of various features, for the historical intermediate prediction data. As another example the training service cleans and prepares the data for training the model. Some examples of cleaning the data include: identifying data that is not formatted properly and removing such data, identifying data with missing aspects and removing the data or updating the missing aspects with default values, identifying features that contain single or very few values and removing such values, identifying and removing duplicate data, and identifying and removing features that have very low correlation to the result. At block 458, the training service receives the feature vectors and uses them to train one or more trained intermediate prediction ML models 114A-N.

In an embodiment, at block 454 the training service also collects additional intermediate prediction data (e.g., data generated from caregiver satisfaction surveys or other human evaluations). At block 456, the training service can also pre-process this additional intermediate prediction data. For example, the feature vectors corresponding to the historical intermediate prediction data can be further annotated using the additional intermediate prediction data. Alternatively, or in addition, additional feature vectors corresponding to the additional intermediate prediction data can be created. At block 458, the training service uses the pre-processed additional intermediate prediction data during training to generate the trained one or more intermediate prediction ML models 114A-N.

In an embodiment, while a variety of suitable data is available for the training service (e.g., the historical intermediate prediction data discussed with regard to block 452 and the additional intermediate prediction data discussed with regard to block 454), a subset of this data is selected to use for training of the one or more intermediate prediction ML models. That is, as part of model design the training data is selected from an available universe of training data. Further, the one or more intermediate prediction ML models can then use the same, or similar, data types or fields for inference for inference (e.g., as discussed above with regard to FIG. 4A). This is also true of training the retention ML model 124, as discussed below with regard to FIG. 8B. Each of the supervised ML models can be trained using a selected subset of data types and fields, and the same or similar data types and fields can be used for inference.

In an embodiment, the pre-processing and training can be done as batch training. In this embodiment, all data is pre-processed at once (e.g., all historical intermediate prediction data and additional intermediate prediction data), and provided to the training service at block 458. Alternatively, the pre-processing and training can be done in a streaming manner. In this embodiment, the data is streaming, and is continuously pre-processed and provided to the training service. For example, it can be desirable to take a streaming approach for scalability. The set of training data may be very large, so it may be desirable to pre-process the data, and provide it to the training service, in a streaming manner (e.g., to avoid computation and storage limitations). Further, in an embodiment, a federated learning approach could be used in which multiple healthcare entities contribute to training a shared model.

Further, in an embodiment, the training can be performed by dividing input data into a training set and a test set. The training set can include a majority of the data (e.g., 80%), and the test set can include the remaining data. Further, the training set itself can be split into two groups: a training group and a validation-on-training group (e.g., split 70/30). The test and validation on training data can be used to predict the trained model's performance under real world conditions.

### Example of Intermediate Prediction Characteristics

FIG. 5A depicts example facility data 500 for determining a facility score using ML, according to one embodiment. In an embodiment, the facility data 500 provide examples for facility characteristics used to generate a facility score, as discussed above in relation to block 352 illustrated in FIG. 3B. In an embodiment, a facility 502 can describe a residential healthcare facility, and outpatient healthcare facility, an in-home healthcare facility (e.g., a patient's home), or any other suitable facility.

In an embodiment, each facility 502 includes facility attributes 510. The facility attributes 510 include a management attribute 512. For example, the management attribute 512 can describe characteristics of the management of the facility. This can include management experience (e.g., total or average experience for managing employees), a management structure (e.g., on-site management, remote management, hybrid management, or any other suitable management structure), a size of management, a measure of management turnover (e.g., over time), a measure of employee turnover (e.g., caregiver and administrative staff turnover), management popularity (e.g., based on caregiver or other employee surveys), management retention rate (e.g., historical turnover rate for caregivers reporting to management), or any other suitable management attributes. Further, the management attribute 512 can describe schedule and task disparities between employees. As discussed further below with regard to FIG. 7, some tasks may be more burdensome and unpleasant and may contribute more to employee turnover. Disparities in assignment of these tasks (e.g., requiring some caregivers to perform the tasks more than other caregivers) can lead to caregiver turnover. The management attribute 512 can describe any disparities in task assignments and scheduling.

The management attribute 512 can further include an onboarding attribute 513. The onboarding attribute 513 can include caregiver ongoing characteristics, including permitted new caregiver ramp up time, number or type of training offered, timing of patient assignment, and any other suitable onboarding characteristics.

The facility attributes 510 can further include a resources attribute 514. For example, the resources attribute 514 can describe available resources at the facility. This can include software resources (e.g., patient management software used, caregiver assistance software used, enterprise business software used, or any other suitable software resources), patient care resources (e.g., available patient care equipment and technology), caregiver resources (e.g., training and assistance available to caregivers), administrative resources (e.g., available administrative support and approval processes required for electronic health records) and any other suitable resources.

The resources attribute 514 can further include an electronic systems attribute 515. The electronic systems attribute 515 can include characteristics of usage of electronic systems by caregivers. For example, the electronic systems attribute 515 can record requests for assistance with electronic software by caregivers, caregiver complaints with electronic software, caregiver usage rates of electronic software, and any other suitable data.

The facility attributes 510 can further include a density attribute 516. For example, the density attribute 516 can describe a density of the facility, in terms of patients and required tasks. For example, a facility that is large in size (e.g., in square footage), but with relatively few patients, may require a caregiver to walk long distances between patients. Further, a facility that is large in size may require a caregiver to walk long distances in performing a task (e.g., to walk between a patient room and a storage facility, cleaning facility, or toilet facility). This could be detrimental to caregiver retention. The density attribute 516 can measure the required walking (or other on-job travel) required by a caregiver. The density attribute 516 can further measure patent turnover (e.g., a frequency or rate of patient turnover).

The facility attributes 510 can further include a compensation and amenities attribute 518. For example, the compensation and amenities attribute 518 can describe the caregiver compensation (e.g., average compensation, absolute compensation, spread of compensation, or any other aspect of caregiver compensation), both in terms of amount and type (e.g., hourly, daily, salaried). The compensation can be described in absolute terms, or in relative terms compared to the location of the facility or regional or national averages (e.g., a percentile above or below local or regional norms).

As another example, the compensation and amenities attribute 518 can describe amenities available to caregivers. This can include compensation related amenities (e.g., available sick time and vacation time, retirement or pension offerings, family leave, or corporate discounts), caregiver health plan amenities (e.g., providing health insurance to caregivers), and other outside of work amenities (e.g., childcare offerings or discounts, concierge services, mental health services, social activities, sporting event or performance tickets or other amenities). The amenities can further include at-work amenities, including meal availabilities (e.g., an on-site cafeteria or restaurant), break rooms or social areas, outside areas, rest or napping areas, seating areas, gym or workout areas, caregiver recognition and celebration, or other at-work amenities. The amenities can be expressed textually, or using a numeric system. The compensation and amenities 518 can further describe career growth for caregivers at the facility (e.g., promotion rates, promotion frequency, and any other suitable data). Further, the compensation and amenities attribute 518 can describe the results of caregiver or employee surveys relating to amenities (e.g., satisfaction surveys). These are merely examples, and the facility attributes 510 can include any suitable characteristics.

In an embodiment, each facility 502 further includes a location 520. For example, the location 520 can describe a geographic location of the facility. Further, the location 520 can describe a proximity of the facility to a population center (e.g., a nearby city or town), a population density of the facility location, and any other suitable location data. In an embodiment, a facility located distant from population centers could tend to increase caregiver turnover (e.g., due to potentially longer commutes for caregivers). Alternatively, or in addition, a facility located in an area with a higher population density could also tend to increase caregiver turnover (e.g., due to available alternative employment options for caregivers). The location 520 can describe information relating to the job market in the relevant area (e.g., a number of caregiver job openings in the geographic region). The location 520 can further describe state and local regulations relating to the facility. For example, publicly available data can be used to generate a dictionary of state and local regulations applicable to facilities in different locations. These are merely examples, and the facility 502 can include any suitable data.

The facility 502 can further include historical record attribute 522. The historical record attribute 522 can indicate a historical outcome rate (e.g., rate of positive and negative patient outcomes), patient transfer rate, patient fall rate, and any other suitable patient information. For example, the historical record attribute 522 can describe a historical rate of falls for patients at a facility, a historical rate of significant injuries from falls for patients at a facility, or any other suitable data.

FIG. 5B depicts example patient data 530 for determining a patient score using ML, according to one embodiment. In an embodiment, the patient data 530 provide examples for patient characteristics used to generate a patient score, as discussed above in relation to block 354 illustrated in FIG. 3B.

In an embodiment, each patient 532 includes patient characteristics 540. The patient characteristics 540 include a number and age 542. For example, the number and age 542 can describe a number of patients cared for at a given facility. For a residential facility, the number can describe the number of patients resident at the facility. For a non-residential facility, the number can describe the number of patients cared for over a given time period (e.g., hourly, daily, weekly, or any other suitable time period). In an embodiment, the number can describe an average (e.g., mean, median, or mode) number of patients for the facility, over a given time period. Further, the number and age 542 can describe an age of patients cared for at the facility. This can include an age statistic (e.g., mean, median, or mode), an age range, or any other suitable age description.

The patient characteristics 540 can further include an outcomes attribute 544. In an embodiment, the outcomes attribute 514 can describe patient outcomes at the facility (e.g., treatment time, recovery outcomes, and any other suitable outcome data).

The patient 532 can further include a diagnoses attribute 550. The diagnoses attribute can include characteristics of patient diagnoses, for a given patient or group of patients (e.g., patients at a given facility). For example, the diagnoses attribute 550 can record diagnosis codes for a given patient, or group of patients.

The diagnoses attribute 550 can include an acuity attribute 552 that describes an acuity (e.g., an intensity of required care) for a given patient or group of patients. The acuity can describe a particular patient's acuity (e.g., a patient for whom a relevant caregiver is caring), an average patient acuity (e.g., mean, median, or mode) for patients at a given facility, a range of acuity (e.g., from least severe to most severe) for patients at the facility, or any other suitable description. Further, the acuity attribute 552 can describe changes in acuity over time (e.g., increases or decreases in patient acuity for a given caregiver, group of caregivers, facility, or group of facilities).

The diagnoses attribute 550 can further include a POC indicators attribute 554. The POC indicators attribute 554 can describe any examination or analysis of the patient as part of POC documentation. This can include identifications of patient behavior issues, memory care, eating behavior, levels of patient assistance required, abusive behavior by patients, and any other suitable information.

The diagnosis attribute 550 can further includes a vitals attribute 556. The vitals attribute 556 can include any suitable information about patient vitals, including vitals values, frequency of recording of vitals, comparisons of patient vitals with baseline patient vitals, any other suitable information.

The diagnosis attribute 550 can further include a minimum data set (MDS) attribute 558. In an embodiment, MDS provides screening and assessment data for a patient or group or patients. The MDS attribute 558 can describe changes in MDS (e.g., a number of significant MDS changes for patients), MDS values, or any other suitable data.

The patient 533 can further include a medications attribute 560. The medications attribute can include a number of medications prescribed or provided to a given patient or group of patients, a number of patients provided with a relatively high number of medications (e.g., more than 10 medications), a record of adverse reactions to medications, a frequency of medication, a number of late administrations of medication (e.g., including any reasons for the error), a number of missed administrations of medication (e.g., including any reasons for the error), a number of amended medication records submitted, and any other suitable data.

In an embodiment, any of the patient characteristics can be determined automatically. For example, an electronic health record for a patient, caregiver clinical notes relating to a patient, or any other suitable textual description, can be analyzed (e.g., using NLP techniques) to determine the patient characteristics. This is discussed further, below, with regard to FIG. 10.

Further, the patient characteristics (e.g., patient acuity and outcomes) can be described numerically (e.g., on a scale from 0-1, 1-10, 1-100, or any other suitable numeric scale), textually (e.g., using textual labels to describe patient acuity), or using any other suitable technique. These are merely examples, and the patient characteristics 530 can include any suitable characteristics.

FIG. 5C depicts example caregiver performance data 570 for determining a caregiver score using ML, according to one embodiment. In an embodiment, the caregiver performance data 570 provide examples for caregiver performance characteristics used to generate a caregiver performance score, as discussed above in relation to block 356 illustrated in FIG. 3B.

In an embodiment, each caregiver performance 572 includes a caregiver errors attribute 580. The caregiver errors attribute 580 describes errors by a given caregiver, or group of caregivers (e.g., caregivers at a given facility). For example, the caregiver errors attribute 580 can include a missed documentation attribute 582. The missed documentation attribute 582 can indicate a frequency of missed documentation by caregivers, a quantity of missed documentation by caregivers, a type of missed documentation by caregivers, and any other suitable data.

The caregiver errors attribute 580 can further include a judgment errors attribute 584. The judgment errors attribute 584 can describe judgment errors by a given caregiver or group of caregivers, including errors in identifying critical situations and contacting emergency or physician services and any other suitable errors. The judgment errors attribute 584 can further include a hospital readmissions attribute 586, reflecting hospital readmissions for a given patient or group of patients (e.g., a rate of readmissions, quantity of readmissions, or any other suitable data).

The caregiver errors attribute 580 can further include a reprimands attribute 588. The reprimands attribute 588 can describe reprimands for a given caregiver or group of caregivers (e.g., caregivers at a given facility). This can include reprimands from a supervisor, a licensing board, or any other suitable source.

The caregiver performance attribute 572 can further include a work characteristics attribute 590. The work characteristics attribute 590 can describe characteristics of work for a given caregiver or group of caregivers. This can include a shift schedule attribute 592 (e.g., describing a shift schedule for a caregiver or group of caregivers). For example, the shift schedule attribute 592 can describe a daily shift average, changes to shifts, shift length, or any other suitable data. The work characteristics attribute 590 can further include a number of facilities attribute 594 (e.g., describing a number of facilities worked at for a given caregiver or group of caregivers), an accolades attribute 596 (e.g., describing accolades for a given caregiver or group of caregivers, including a rate of accolades, number of accolades, source of accolades, or any other suitable data), a traumatic events attribute 598 (e.g., describing patient deaths or other potentially traumatic events for a given caregiver or group of caregivers), or any other suitable data.

FIG. 6 depicts example progress note data 600 for determining a progress note score using ML, according to one embodiment. In an embodiment, the caregiver progress note data 600 provide examples for progress note characteristics used to generate a progress note score, as discussed above in relation to block 358 illustrated in FIG. 3B.

In an embodiment, each progress note 602 includes metadata 610. The metadata 610 describes metadata characteristics of a progress note, or group of progress notes. The metadata 610 can include any suitable progress note data, including a length 612 (e.g., describing a number of words, number of characters, number of topics, or any other suitable length data for a progress note or group of progress notes), an entry time 614 (e.g., describing a time at which a progress note or group of progress notes is entered), and an entry duration 616 (e.g., describing a time duration needed for a caregiver to enter a progress note or group of progress notes).

In an embodiment, the progress note 602 (e.g., the entry time 614 and entry duration 616) can be used to identify overtime worked by a caregiver or group of caregivers (e.g., based on an expected shift schedule) and to generate a timeline of caregiver work. This can further be used in generating a progress note score, as described above.

The progress note 602 further includes a text characteristics attribute 620. For example, the text characteristics attribute 620 can describe characteristics of the text entered into the progress note or group of progress notes. This can include a word usage attribute 622 (e.g., a level of sophistication of words used, reading level of words used, length of words used, or any other suitable data), or any other text characteristics. In an embodiment, NLP techniques can be used to analyze the progress notes and automatically identify the text characteristics. This is discussed further, below, with regard to FIG. 10.

The progress note 602 further includes a frequency characteristics attribute 630. In an embodiment, the frequency characteristics attribute 630 identifies characteristics of the frequency with which a caregiver or group of caregivers enter progress notes. This can include any suitable frequency characteristic data, including a number of notes attribute 632 (e.g., a number of notes entered by a caregiver, a number of notes or average number of notes entered by a group of caregivers, or any other suitable data) and a missing notes attribute 634 (e.g., a quantity or frequency of expected, but missing, progress notes for a caregiver or group of caregivers).

FIG. 7 depicts example task data 700 for determining a task score using ML, according to one embodiment. In an embodiment, the task data 700 provide examples for task characteristics used to generate a task score, as discussed above in relation to block 360 illustrated in FIG. 3B.

In an embodiment, each task 702 relates to one more tasks for a caregiver to perform as part of caring for a patient. Each task 702 includes task attributes 710. For example, the task attributes 710 can describe attributes of the task. The task attributes 710 can include binary attributes (e.g., whether the task involves particular aspects of caregiving, including toileting a patient, wound care for a patient, lifting a patient, washing a patient, or any other suitable aspect). The task attributes 710 can further describe a training or skill level required for the task (e.g., a required academic degree, certification, or years of experience), a required physical ability level (e.g., an ability to lift a required weight), or any other attribute of the task. These are merely examples, and the task attributes 710 can include any suitable characteristics.

The task 702 can further include survey results 720. For example, the survey results 720 can indicate the results of surveys given to caregivers. The survey results 720 can be expressed numerically (e.g., an average score given by caregivers, a standard deviation of scores given by caregivers, a range of scores given by caregivers, a response rate by caregivers, or any other suitable numeric description) or textually. The survey results 720 can include a difficulty 722. For example, the difficulty 722 can describe a perceived difficult of the task to caregivers, expressed in the survey results.

The survey results 720 can further include a time 724. For example, the time 724 can describe a required time for the task, as expressed in the survey results. The time 724 can be expressed as a numeric value (e.g., a number of minutes) or as a function of the allotted time. For example, a particular task could be expected to take a particular duration (e.g., 1 hour), but caregivers could express that the task actually takes significantly more, or less, than the allotted time. The time 724 could describe this ratio (e.g., a percentage of allotted time taken for the task).

The survey results 720 can further include a burden 726. For example, the burden 726 can describe the physical burden of performing the task, as expressed in the survey results. As one example, a task requiring lifting a patient could be more physically burdensome than a task relating to providing a patient with medication. As another example, the burden 726 can describe the mental or emotional burden of performing the task, as expressed in the survey results. As one example, a complex task could be more mentally burdensome than a simple task, and a task at a hospice facility could be more emotionally burdensome than a task at a facility with low patient acuity. The burden 726 can be expressed numerically (e.g., using a tuple or a single number expressing burden on a numeric scale), textually, or using any other suitable technique. These are merely examples, and the survey results 720 can include any suitable data.

Further, in an embodiment, the difficulty 722, time 724, burden 726, or any other suitable attributes can be determined automatically (e.g., by parsing textual data), without a caregiver survey, or in addition to using a caregiver survey. For example, caregiver notes, task descriptions, or other textual description, relating to the task can be analyzed (e.g., using NLP techniques) to determine the content of the textual description. This can be used to determine the difficulty 722, time 724, burden 726, or any other suitable attributes for the task. This is discussed further, below, with regard to FIG. 10.

In an embodiment, the task 702 further includes a distance traveled 740. For example, the distance traveled 740 can describe the distance that a caregiver must travel to perform the task (e.g., a distance that a caregiver must walk within a facility to perform the task). This could be measured (e.g., using a fitness tracker carried by a caregiver, tracking of caregiver credentials through a facility, image recognition using suitable image capture devices, or using any other suitable techniques), pre-defined (e.g., by a management employee creating or describing the task), derived from a survey, or determined using any other suitable technique. The distance traveled can be expressed numerically as an absolute value (e.g., in feet, meters, or steps traveled), numerically as a relative value (e.g., relative to other tasks performed by caregivers), textually, or using any other suitable technique. These are merely examples, and the task 702 can include any suitable data.

### Example of Predicting Retention Data

FIG. 8A illustrates predicting caregiver retention using an ML model, according to one embodiment. In an embodiment, FIG. 8A provides one example of predicting retention data for a given caregiver, or group of caregivers, using an ML model, as discussed above in relation to block 3084 illustrated in FIG. 3. Intermediate prediction scores 410A-N (e.g., as discussed above in relation to FIG. 4A) are provided to a retention service 122 and a retention ML model 124. In an embodiment, the intermediate prediction scores 410A-N indicate predicted impact of various intermediate prediction characteristics on retention of a given caregiver or group of caregivers. For example, as discussed above in relation to FIG. 4A, each of the intermediate prediction scores 410A-N can include a tuple indicating a predicted likelihood that the relevant characteristic contributes to retention or turnover, and a predicted magnitude of the contribution.

In an embodiment, caregiver data 182 is also provided to the retention service 122 and the retention ML model 124. For example, the caregiver data 182 can describe characteristics of the caregiver, or group of caregivers. This can include the caregiver characteristics 920 illustrated below in relation to FIG. 9 (e.g., demographics, status, performance, compensation, and any other suitable characteristics). The caregiver data 182 can further includes compatibility attributes for the caregiver(s) and facilities (e.g., compatibility attributes 930 illustrated below in relation to FIG. 9). The compatibility attributes can include commute attributes, staffing attributes, and any other suitable compatibility attributes. These are merely examples, and the caregiver data can include any suitable data.

In an embodiment, compatibility data 184 is also provided to the retention service 122 and the retention ML model 124. For example, the compatibility data 184 can describe compatibility characteristics between a caregiver and facility (e.g., commute, staffing, and any other suitable data). This is described further, below, with regard to FIG. 9.

The compatibility data 184 can further include external characteristics relating to the caregiver. This can include employment and economic information for the caregiver's local region (e.g., unemployment rates, cost of living information, average compensation, and any other suitable information), demographic information for the caregiver's local region (e.g., average ages for the region, education levels for the region, and any other suitable information), other jobs available in the caregiver's local region (e.g., type of job, compensation offered, skill required, or any other suitable data). The compatibility data 184 can further include store locator data and survey data describing other available employment options. Thus, store locator data may be imported from mapping solutions and include the number of stores or businesses open in a specific location. The data can be used to calculate the number of competitive job opportunities close to the caregiver's home location (e.g., determined using a global positioning system (GPS) system). These are merely examples, and the retention ML model 124 can receive any subset of the intermediate prediction scores 410A-N, the caregiver data 182, the compatibility data 184, or any other suitable data. In an embodiment, outcome data (e.g., data describing prior employment outcomes for a given caregiver or group of caregivers, as described below in relation to FIG. 9) can also be provided to the retention service 122 and retention ML model 124.

In an embodiment, the caregiver data 182, the compatibility data 184, or any other suitable data (e.g., outcome data 940 illustrated below in FIG. 9), can be analyzed using a suitable ML model, and an intermediate prediction score relating to the data can be provided to the retention service 122. These one or more intermediate prediction scores relating to the caregiver data 182, the compatibility data 184, or other suitable data, can be provided to the retention service 122 and retention ML model 124 in place of, or in addition to, the data itself.

In an embodiment, the retention service 122 can use the retention ML model 124 to predict any, or all, of a predicted retention 812, retention factors 814, and retention recommendations 816 (e.g., during inference). For example, the predicted retention 812 can indicate a likelihood that a given caregiver, or group of caregivers, will be retained or will turnover. In an embodiment, the predicted retention 812 is a number, or tuple of numbers, indicating the likelihood that a given caregiver, or group of caregivers, will be retained or will turnover. This is merely an example, and the task score can be any suitable value or collection of values (e.g., one or more Boolean values, textual values, or any other suitable values).

As another example, the retention factors 814 can predict a collection of factors that are likely to be contributing to the caregiver(s) retention or turnover. In an embodiment, the retention factors 814 identify one or more likely factors contributing to retention or turnover. For example, the retention ML model 124 can predict which of the input factors (e.g., one or more of the intermediate prediction scores 410A-N, one or more attributes of the caregiver data 182 or compatibility data 184, or both) are likely to contribute to retention or turnover for the caregiver(s). In an embodiment, the retention factors 814 are numbers, or tuples of numbers, indicating one or more of likelihood and magnitude that a given factor contributes to retention or turnover. For example, the retention factors 814 can include tuple of two numbers, for each identified factor, indicating the likelihood that the factor contributes to turnover (or retention) and the magnitude of the contribution. This is merely an example, and the retention factors 814 can be any suitable value or collection of values (e.g., one or more Boolean values, textual values, or any other suitable values). In an embodiment, the retention ML model 124 identifies a primary retention factor, among a group of retention factors 814, that the ML model predicts is the most significant retention factor.

Further, in an embodiment, the retention factors can be provided in a suitable user interface to assist in improving retention. For example, a user interface can identify types of caregivers at risk for turnover, specific caregivers predicted to be at risk for turnover (e.g., by providing identifying information for specific caregivers), a number of patients managed by caregivers at risk for turnover (e.g., presented using a graph depicting changes in number of patients managed over time), changes in progress notes for caregivers at risk for turnover (e.g., identifying aspects or portions of progress notes suggesting a risk of turnover), shifts for caregivers at risk for turnover (e.g., a graph identifying changes in shifts over time), or any other suitable information.

As another example, the retention recommendations 816 can predict a collection of recommendations that are likely to improve retention. In an embodiment, the retention factors 814, discussed above, identify one or more likely factors contributing to retention or turnover. The retention recommendations 816 can use the identified factors to predict recommendations. For example, the retention ML model can be trained to identify changes to each factor that improve retention. As another example, the retention ML model can be provided with a dictionary providing a suggested recommendation to improve retention, given a particular factor (e.g., a pre-defined dictionary providing suggested recommendations for known potential factors). For example, caregiver employment status (e.g., part-time) could be identified as a factor leading to turnover, and the retention recommendations 816 could recommend offering the caregiver additional employment hours or full-time status. As another example, task difficulty could be identifies as a factor leading to turnover, and the retention recommendations 816 could recommend switching employees performing difficult tasks, offering additional compensation for difficult tasks, rotating employees required to perform difficult tasks, or any other suitable recommendation.

In an embodiment, the retention ML model 124 can be any suitable ML model, or group of ML models. For example, a suitable ML model could be used to predict each of the predicted retention 812, retention factors 814, and 816. In an embodiment, the retention ML model 124 can include multiple ML models that depend on each other for input. For example, one ML model could be used to identify retention factors 814, and the output of that model could be used as the input to another ML model to identify retention recommendations 816. This is merely an example. In an embodiment, one or more neural networks can be used (e.g., a DNN or any other suitable neural network) or one or more non neural-networks can be used (e.g., a logistic regression model, a linear regression model, a decision tree, a support vector machine, a Bayesian network, a gradient boosting machine, or any other suitable non-neural network ML model). Further, in an embodiment, the retention ML model 124 could include multiple ML models with different types and structures (e.g., different ML models to predict each of the predicted retention 812, the retention factors 814, and the retention recommendations 816).

While FIG. 8A illustrates using a retention ML model 124, in an embodiment the retention service can generate any, or all, of the predicted retention 812, retention factors 814, and retention recommendation 816 without using an ML model. For example, one or more intermediate prediction ML models can be used to generate the intermediate prediction scores 410A-N, and the retention service 122 can use these scores (and any other suitable data) to generate the predicted retention 812, retention factors 814, and retention recommendation 816 without using an ML model.

In an embodiment, the retention service can further prophylactically identify and cure an incompatibility between a caregiver and patient, facility, or task. For example, the retention service can use patient medical data, including but not limited to specific health related data associated with one or more patients, such as age, weight, medical conditions, demographics, or other such data, along with one or more of intermediate prediction characteristics (e.g., intermediate prediction characteristics 132 illustrated in FIG. 1) and caregiver data 182, to identify a likely incompatibility between the patient and the caregiver. As one example, a patient could be identified as requiring a particular treatment task or collection of treatment tasks, which is likely to lead to turnover for the caregiver. The retention service 122 can transmit an alert (e.g., an e-mail, SMS message, telephone call, or another form of electronic message) describing the incompatibility to a healthcare facility (e.g., a healthcare facility 1160 illustrated in FIG. 11) or care provider (e.g., a care provider 1150 illustrated in FIG. 11, including a management employee for a facility relating to the patient's treatment). The alert can be used to schedule a different care provider and cure the incompatibility. In an embodiment, the retention service can identify this incompatibility prior to completing the prediction of the predicted retention 812. For example, the retention service can identify a high priority incompatibility while predicting the predicted retention 812, and can transmit the alert prior to completing the prediction of the predicted retention 812. In an embodiment this allows for a rapid alert for the incompatibility, without waiting for complete prediction of the predicted retention.

### Example of Training a Retention ML Model

FIG. 8B is a flowchart 850 illustrating training an ML Model to predict caregiver retention, according to one embodiment. This is merely an example, and in an embodiment another suitable technique could be used (e.g., without requiring training). At block 852, a training service (e.g., a human administrator or a software or hardware service) collects historical caregiver outcome data. For example, a retention service (e.g., the retention service 122 illustrated in FIGS. 1 and 2) can be configured to act as the training service and collect historical caregiver outcome data (e.g., the historical caregiver outcomes 142 illustrated in FIG. 1). The historical caregiver outcome data is discussed further, below, with regard to FIG. 9. This is merely an example, and any suitable software or hardware service can be used (e.g., a retention training service) and any suitable training data can be used.

At block 856, the training service (or other suitable service) pre-processes the collected historical caregiver outcome data. For example, the training service can create feature vectors reflecting the values of various features, for the historical caregiver outcome data. As another example the training service cleans and prepares the data for training the model. Some examples of cleaning the data include: identifying data that is not formatted properly and removing such data, identifying data with missing aspects and removing the data or updating the missing aspects with default values, identifying features that contain single or very few values and removing such values, identifying and removing duplicate data, and identifying and removing features that have very low correlation to the target. At block 858, the training service receives the feature vectors and uses them to train a trained retention ML model 124.

In an embodiment, at block 854 the training service also collects additional historical outcome data. In an embodiment, this includes historical baseline outcome data (e.g., the historical baseline outcomes 144 illustrated in FIG. 1). For example, the historical baseline outcome data can indicate baseline retention outcomes for caregivers (e.g., average turnover for a region, category of caregiver, type of facility, or based on any other criteria).

At block 856, the training service can also pre-process this additional historical outcome data. For example, the feature vectors corresponding to the historical caregiver outcome data can be further annotated using the additional historical outcome data. Alternatively, or in addition, additional feature vectors corresponding to the additional historical outcome data can be created. At block 858, the training service uses the pre-processed additional historical outcome data during training to generate the trained retention ML model 124.

In an embodiment, as described above in relation to FIG. 4B, the pre-processing and training can be done as batch training, in a streaming manner, using federated data, by dividing input data into a training set and a test set, or using any other suitable technique.

FIG. 9 depicts example historical caregiver outcome data 900 for predicting caregiver retention using an ML model, according to one embodiment. In an embodiment, the historical caregiver outcome data 900 provide examples for the historical caregiver outcomes 142, illustrated in FIG. 1. For example, the historical caregiver outcome data 900 can include one or more historical caregiver outcomes 902.

The historical caregiver outcome 902 can include one or more intermediate prediction scores 910A-N (e.g., the intermediate prediction scores 410A-N illustrated in FIG. 4B). As discussed above, in an embodiment the intermediate prediction scores 910A-N indicate a predicted contribution of various characteristics, to retention or turnover of a caregiver or group of caregivers. For example, the intermediate prediction scores can be generated using relevant historical intermediate prediction data (e.g., as described above in relation to FIGS. 3B and 4A), and included as training data.

The historical caregiver outcome 902 further includes one or more caregiver characteristics 920. In an embodiment, the caregiver characteristics 920 include demographics 922. For example, the demographics 922 can include an age of the caregiver, gender, experience level, referral source, educational attainment, certifications, level of training, length(s) of employment, and any other suitable demographic data.

In an embodiment, the caregiver characteristics 920 further include a status attribute 924. For example, the status attribute 924 can describe whether the caregiver is employed full-time, part-time, as a permanent employee, pursuant to a term contract, or any other suitable data. Further, the status attribute 924 can describe whether the caregiver works additional jobs (e.g., for part time caregivers) and if so how many additional jobs and what the additional jobs are (e.g., other caregiver jobs or other categories of jobs). As another example, the status attribute 924 can describe a role of the caregiver (e.g., direct patient care, administration, skilled nursing, physician, or any other suitable role).

The caregiver characteristics 930 can further include a performance attribute 926. The performance attribute 926 can describe the caregiver's job performance (e.g., based on prior employment reviews, patient surveys, or any other suitable data). Further, in an embodiment, the performance attribute 926 can describe a caregiver's satisfaction during their work. For example, a caregiver's notes and description (e.g., staff progress notes, clinical notes, and any other suitable textual description) could be analyzed (e.g., using NLP techniques) to identify the state of mind or satisfaction. This is discussed further, below, with regard to FIG. 10.

The caregiver characteristics can further include a compensation attribute 928. The compensation attribute 938 can describe the caregiver's compensation (e.g., net monetary compensation, gross monetary compensation, amenities, or any other suitable aspect of compensation). These are merely examples, and the caregiver characteristics 920 can include any suitable data.

The historical caregiver outcome 902 further includes one or more compatibility characteristics 930. In an embodiment, the compatibility characteristics 940 describe a compatibility between the caregiver and a given facility, role, or patient population. For example, the compatibility characteristics 930 include a commute attribute 932. The commute attribute 932 can indicate a commute length, or duration, for the caregiver to the facility or facilities where the caregiver works. In an embodiment, this can be calculated automatically (e.g., based on a residence address for the caregiver and an address of the caregiver's workplace(s)).

As another example, the compatibility characteristics 930 further include a staffing attribute 944. In an embodiment, the staffing attribute 934 can indicate a level of staffing for caregivers in the relevant caregiver's role, or for a level of acuity of the patients the caregiver is treating. For example, if the relevant caregiver is a registered nurse, the staffing attribute 934 can indicate a level of staffing (e.g., understaffed for patient needs, overstaffed for patient needs, appropriately staffed for patient needs) for registered nurses at the facility, or facilities, where the caregiver works. As another example, the staffing attribute 934 can indicate a level of staffing for patients with a given level of acuity. These staffing attributes 934 can be compared with baseline values (e.g., for other facilities or caregivers). These are merely examples, and the compatibility characteristics 940 can include any suitable data.

As another example, the compatibility characteristics 904 include an external attribute 936. The external attribute 936 can include external characteristics relating to the caregiver. This can include employment and economic information for the caregiver's local region (e.g., unemployment rates, cost of living information, average compensation, and any other suitable information), demographic information for the caregiver's local region (e.g., average ages for the region, education levels for the region, and any other suitable information), other jobs available in the caregiver's local region (e.g., type of job, compensation offered, skill required, or any other suitable data). This is discussed further, above, with regard to the compatibility data 184 illustrated in FIG. 8A.

The historical caregiver outcome 902 further includes one or more outcome characteristics 940. In an embodiment, the outcome characteristics 940 describe prior employment outcomes for the caregiver. For example, the outcome characteristics 940 can include a duration attribute 942. The duration attribute 942 can indicate a duration at which the caregiver worked at a particular facility or group of facilities (e.g., a length of time in months, weeks, or years). As another example, the outcome characteristics 940 can include a reason attribute 944. The reason attribute 944 can indicate a reason that the caregiver left their prior position (e.g., a stated reason in an exit interview or survey). These are merely examples, and the outcome characteristics 940 can include any suitable data. Further, the historical caregiver outcome data 902 can include any suitable data.

### Pre-Processing Textual Data

FIG. 10 depicts pre-processing textual data using NLP, according to one embodiment. At block 1002, a retention service (e.g., the retention service 122) identifies textual data (e.g., unstructured textual data). The retention service is merely an example, and any suitable software or hardware service or technique can be used.

For example, as discussed above, textual data can be analyzed and used for many different aspects of predicting caregiver retention. As one example, textual data in progress notes can be used to determine progress note characteristics (e.g., the progress note characteristics 600 illustrated in FIG. 6) and generate a progress note score (e.g., as described in relation to block 358 illustrated in FIG. 3B). Further, textual data can be used to determine patient acuity (e.g., patient acuity 552 and outcomes 544 illustrated in FIG. 5B) as part of predicting a patient score. As another example, textual data can be used to determine various task attributes (e.g., a difficulty 722, time 724, or burden 726 as illustrated in FIG. 7), instead of or in addition to a caregiver survey, as part of predicting a task score. As another example, textual data can be used to determine a caregiver's state of mind or satisfaction (e.g., as part of a performance attribute 936 illustrated in FIG. 9) as part of predicting retention data. These are merely examples, and textual data can be used for a variety of purposes as part of predicting caregiver retention.

At block 1004, the retention service identifies an NLP technique to process the textual data. In an embodiment, various NLP techniques can be used to extract meaning from textual data. These techniques can include keyword extraction, named entity recognition, topic modeling, summarization, sentiment analysis, and any other suitable NLP techniques. For example, different NLP techniques can be selected based on the source of the textual data and the desired use for the analysis. In an embodiment, the retention service can identify the source of the textual data and the use for the analysis, and can select a suitable NLP technique.

At block 1006, the retention service analyzes the textual data using NLP. For example, the retention service can use NLP (e.g., a text analytics engine) to analyze a variety of textual data (e.g., as described above in relation to block 1002). These are merely examples, and the retention service can analyze a variety of textual data for a variety of purposes as part of predicting caregiver retention.

### Retention Predictions

FIG. 11 depicts predicted caregiver retention outcomes using ML, according to one embodiment. In an embodiment, a prediction controller 1110 (e.g., the prediction controller 200 illustrated in FIG. 2) generates predicted retention data 1120. For example, as discussed above in relation to FIG. 8A, a retention service (e.g., the retention service 122 illustrated in FIGS. 1-2 and 8A) can use a retention ML model (e.g., the retention ML model 124 illustrated in FIGS. 1-2 and 8A) to predict any, or all, of a predicted retention 812, retention factors 814, and retention recommendations 816.

In an embodiment, the prediction controller 1110 transmits the predicted retention data 1120 over a communication network 1130 to any of, or all of, a care provider 1150 and a healthcare facility 1160. The communication network 1130 can be any suitable communication network, including the Internet, a wide area network, a local area network, or a cellular network, and can use any suitable wired or wireless communication technique (e.g., WiFi or cellular communication).

In an embodiment, any, or all, of the care provider 1150 and the healthcare facility 1160 receive the predicted retention data 1120. The predicted retention data 1120 can then be used to improve and plan for caregiver retention, thereby improving patient care and outcomes. For example, the care provider 1150 or the healthcare facility 1160 (e.g., a) can receive the predicted retention data 1120. The care provider 1150 or healthcare facility 1160 (e.g., an electronic system at the care provider 1150 or healthcare facility 1160) can use the predicted retention data 1120 to improve retention (e.g., implementing retention recommendations 816 or using retention factors 814 to identify improvements). For example, the care provider 1150 or healthcare facility 1160 (e.g., an electronic system at the care provider 1150 or healthcare facility 1160) can present the predicted retention data 1120 using a suitable user interface, as described further above with regard to FIG. 8A.

Further, the care provider 1150 or healthcare facility 1160 can use the predicted retention data 1120 (e.g., the predicted retention 812) to ensure that sufficient resources and staffing are available for the patient. For example, the care provider 1150 or healthcare facility 1160 can use the predicted retention data 1120 to plan for possible turnover, and ensure that the patients receive the necessary staffing from caregivers.

In an embodiment, the prediction controller 1110 can interact directly with a care provider 1150 or healthcare facility 1160 to improve retention. For example, the prediction controller 1110 can interact directly with electronic systems of a care provider 1150 or healthcare facility 1160 (e.g., using a suitable application programming interface (API), web interface, or other electronic interface) to implement retention recommendations 816. This can include scheduling caregivers for different tasks or facilities, upgrading or improving electronic systems, scheduling caregivers to improve coverage or change management structures, scheduling caregivers to reduce the individual burden of difficult or unpleasant tasks, and implementing any other suitable improvements. In one embodiment, the care provider 1150 or healthcare facility 1160 implements some, or all, of the retention recommendations 816 automatically. Alternatively, or in addition, the care provider 1150 or healthcare facility 1160 provides the retention recommendations to a suitable administrator, or administrators, for consideration and implementation.

In an embodiment, the prediction controller 1110 can interact directly with a staffing system to schedule and hire caregivers. For example, the prediction controller 1110 can interact directly with a scheduling system of a healthcare facility 1160 (e.g., using a suitable application programming interface (API), web interface, or other electronic interface) to schedule caregivers to cover any potential turnover. As another example, the prediction controller 1110 can interact directly with a hiring system of a healthcare facility 1160 (e.g., using a suitable application programming interface (API), web interface, or other electronic interface) to place hiring announcements or suggestions to cover any potential turnover.

In an embodiment, any, or all, of the care provider 1150, and the healthcare facility 1160 store the predicted retention data 1120. For example, this can allow the recipient to access the predicted retention data 1120 without requiring a continuous network connection.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in FIGS., those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
predicting an impact of one or more caregiver tasks on continued employment of the caregiver with a care provider, comprising:
determining a plurality of intermediate prediction scores relating to characteristics for the caregiver using one or more first machine learning (ML) models trained to determine intermediate prediction scores; and
determining a retention prediction for the caregiver using the plurality of intermediate prediction scores, comprising:
generating the retention prediction by providing the plurality of intermediate prediction scores to a second ML model trained to determine the retention prediction based on intermediate prediction scores,
wherein the retention prediction is provided to an electronic system relating to the caregiver to improve treatment for a patient of the caregiver by at least one of: (i) increasing a likelihood of continued employment for the caregiver or (ii) identifying a replacement for the caregiver.

2. The method of claim 1, wherein each of the plurality of intermediate prediction scores relate to at least one of: (i) a facility score, (ii) a patient score, (iii) a caregiver performance score, (iv) a progress note score, or (v) a task score.

3. The method of claim 2, further comprising:
predicting a most impactful intermediate prediction score, among the plurality of intermediate prediction scores, to continued employment of the caregiver.

4. The method of claim 2, wherein the one or more first ML models comprises a plurality of first ML models, each of the plurality of first ML models trained to determine one of the plurality of intermediate prediction scores.

5. The method of claim 1, wherein the retention prediction comprises at least one of: (i) a likelihood that the caregiver will continue employment with the care provider for a period of time, (ii) one or more factors predicted to impact the likelihood that the caregiver will continue employment with the care provider, or (iii) one or more recommended actions predicted to improve the likelihood that the caregiver will continue employment with the care provider.

6. The method of claim 5, wherein the retention prediction comprises all of: (i) a likelihood that the caregiver will continue employment with the care provider for a period of time, (ii) one or more factors predicted to impact the likelihood that the caregiver will continue employment with the care provider, and (iii) one or more recommended actions predicted to improve the likelihood that the caregiver will continue employment with the care provider.

7. The method of claim 6, wherein the second ML model comprises a plurality of different ML models, each of the plurality of different ML models trained to determine one of the: (i) likelihood that the caregiver will continue employment with the care provider for a period of time, (ii) one or more factors predicted to impact the likelihood that the caregiver will continue employment with the care provider, and (iii) one or more recommended actions predicted to improve the likelihood that the caregiver will continue employment with the care provider.

8. The method of claim 1, further comprising:
identifying a prophylactic incompatibility between the caregiver and at least one of a patient, a healthcare facility, or a caregiver task; and
transmitting an electronic alert relating to the incompatibility.

9. The method of claim 8, wherein identifying the prophylactic incompatibility further comprises:
transmitting the alert electronically using a communication network, prior to completing the determining the retention prediction.

10. The method of claim 1, further comprising:
identifying textual data for use by at least one of the first ML model or the second ML model; and
pre-processing the textual data using natural language processing (NLP) prior to providing the textual data to a respective ML model.

11. An apparatus comprising:
a memory; and
a hardware processor communicatively coupled to the memory, the hardware processor configured to perform operations comprising:
predicting an impact of one or more caregiver tasks on continued employment of the caregiver with a care provider, comprising:
determining a plurality of intermediate prediction scores relating to characteristics for the caregiver using one or more first machine learning (ML) models trained to determine intermediate prediction scores; and
determining a retention prediction for the caregiver using the plurality of intermediate prediction scores, comprising:
generating the retention prediction by providing the plurality of intermediate prediction scores to a second ML model trained to determine the retention prediction based on intermediate prediction scores,
wherein the retention prediction is provided to an electronic system relating to the caregiver to improve treatment for a patient of the caregiver by at least one of: (i) increasing a likelihood of continued employment for the caregiver or (ii) identifying a replacement for the caregiver.

12. The apparatus of claim 11, wherein each of the plurality of intermediate prediction scores relate to at least one of: (i) a facility score, (ii) a patient score, (iii) a caregiver performance score, (iv) a progress note score, or (v) a task score.

13. The apparatus of claim 12, wherein the one or more first ML models comprises a plurality of first ML models, each of the plurality of first ML models trained to determine one of the plurality of intermediate prediction scores.

14. The apparatus of claim 13, wherein the second ML model comprises a plurality of different ML models, each of the plurality of different ML models trained to determine one of the: (i) likelihood that the caregiver will continue employment with the care provider for a period of time, (ii) one or more factors predicted to impact the likelihood that the caregiver will continue employment with the care provider, and (iii) one or more recommended actions predicted to improve the likelihood that the caregiver will continue employment with the care provider.

15. The apparatus of claim 11, further comprising:
identifying a prophylactic incompatibility between the caregiver and at least one of a patient, a healthcare facility, or a caregiver task; and
transmitting an electronic alert relating to the incompatibility.

16. The apparatus of claim 15, wherein identifying the prophylactic incompatibility further comprises:
transmitting the alert electronically using a communication network, prior to completing the determining the retention prediction.

17. A non-transitory computer-readable medium comprising instructions that, when executed by a processor, cause the processor to perform operations comprising:
predicting an impact of one or more caregiver tasks on continued employment of the caregiver with a care provider, comprising:
determining a plurality of intermediate prediction scores relating to characteristics for the caregiver using one or more first machine learning (ML) models trained to determine intermediate prediction scores; and
determining a retention prediction for the caregiver using the plurality of intermediate prediction scores, comprising:
generating the retention prediction by providing the plurality of intermediate prediction scores to a second ML model trained to determine the retention prediction based on intermediate prediction scores,
wherein the retention prediction is provided to an electronic system relating to the caregiver to improve treatment for a patient of the caregiver by at least one of: (i) increasing a likelihood of continued employment for the caregiver or (ii) identifying a replacement for the caregiver.

18. The non-transitory computer-readable medium of claim 17, wherein each of the plurality of intermediate prediction scores relate to at least one of: (i) a facility score, (ii) a patient score, (iii) a caregiver performance score, (iv) a progress note score, or (v) a task score.

19. The non-transitory computer-readable medium of claim 17, wherein the one or more first ML models comprises a plurality of first ML models, each of the plurality of first ML models trained to determine one of the plurality of intermediate prediction scores.

20. The non-transitory computer-readable medium of claim 17, wherein the retention prediction comprises all of: (i) a likelihood that the caregiver will continue employment with the care provider for a period of time, (ii) one or more factors predicted to impact the likelihood that the caregiver will continue employment with the care provider, and (iii) one or more recommended actions predicted to improve the likelihood that the caregiver will continue employment with the care provider.
